(19) United States
(12) Patent Application Publication
(10) Pub. No.: US 2017/0049679 A1
(43) Pub. Date: Feb. 23, 2017

(54) ORAL AND DENTAL HYGIENE AND CLEANING AGENTS FOR REDUCING TOOTH DISCOLORATION

(71) Applicant: Henkel AG & Co. KGaA, Duesseldorf (DE)

(72) Inventors: Thomas Welss, Duesseldorf (DE); Nicole Duschek, Duesseldorf (DE); Kristin Miehlich, Wuppertal (DE); Wilfried Howorka, Duesseldorf (DE); Daniela Schmitz, Duesseldorf (DE)

(73) Assignee: Henkel AG & Co. KGaA, Duesseldorf (DE)

(21) Appl. No.: 15/345,851

(22) Filed: Nov. 8, 2016

Related U.S. Application Data
(63) Continuation of application No. PCT/EP2015/056090, filed on Mar. 23, 2015.

(30) Foreign Application Priority Data
May 14, 2014 (DE) 10 2014 209 070.9

(51) Int. Cl. A61K 8/81; A61K 8/24; A61Q 11/00

(52) U.S. Cl. CPC A61K 8/8164; A61Q 11/00; A61K 8/24

(57) ABSTRACT

The invention relates to oral and dental hygiene and cleaning agents, including—with respect to the weight thereof—a) 0.001 to 5 wt % of copolymer(s) comprising at least one structural unit of formula (A1) and at least one structural unit of formula (A2); b) formula (A1), formula (A2), wherein R¹ stands for a (C₁ to C₁₈)-alkyl group, R² stands for a (C₁ to C₆)-alkyl group, M stands for a hydrogen atom or an equivalent of a monovalent or polyvalent cation; and c) 0.1 to 10 wt % of pentasodium triphosphate; which result synergistically in improved cleaning power for surface stains on teeth and in improved prevention of reforming dental calculus, without abrasion of hard dental tissue being increased.

### FIELD OF THE INVENTION

The present invention generally relates to oral and dental hygiene agents having a combination of active substances for gently and effectively cleaning teeth.

### BACKGROUND OF THE INVENTION

Dental cleaning agents are on the market in various forms and are used primarily to clean the tooth surface and to prevent tooth and gum diseases. They usually include a combination of polishing agents, humectants, surfactants, binders, flavoring substances, and fluoride-containing and antimicrobial active substances. In addition to tooth powders, which play a subordinate role because of their increased abrasiveness, dental cleaning agents are offered especially in the form of pastes, creams, and translucent or transparent gels. In the last few years, liquid tooth creams and mouthwashes have also become increasingly important.

More than half of consumers are worried about yellow and discolored teeth, because they regard this as a sign of poor oral hygiene or aging. The desire of customers for dental hygiene products which, in addition to meeting the basic needs (plaque removal and cavity prevention), also have a positive effect on surface tooth discolorations is continuously growing. This is also reflected in the market for dental and oral hygiene. Between 2009 and 2012, the number of products advertising the removal of stains increased by 17%.

Stains on the tooth surface can be caused by the consumption of intensely colored beverages and foods (for example, tea or red wine) or by increased mineralization of plaque, the calculus.

Many products achieve the removal of stains on teeth by using harsh cleaning particles, which can lead to greatly increased abrasive wear of the hard dental tissue. More and more, customers are looking for products that efficiently lead to whiter teeth but are gentle on the hard dental tissue.

The present invention addresses the problem of satisfying this consumer need and providing preparations for oral and dental hygiene and cleaning that achieve effective cleaning and reduction of the discoloration of teeth while preventing or reducing new discolorations for as long as possible.

Interestingly, it has now been found that the combination of specific polymers and pentasodium triphosphate synergistically leads to an improvement in the cleaning performance for surface stains on teeth and to improved reduction of reforming calculus without leading to increased abrasive wear of hard dental tissue, which was unforeseeable to a person skilled in the art.

Furthermore, other desirable features and characteristics of the present invention will become apparent from the subsequent detailed description of the invention and the appended claims, taken in conjunction with this background of the invention.

BRIEF SUMMARY OF THE INVENTION

An oral and dental hygiene and cleaning agent, including—with respect to the weight thereof—0.001 to 5 wt % of copolymer(s), comprising at least one structural unit of formula (A1) and at least one structural unit of formula (A2)

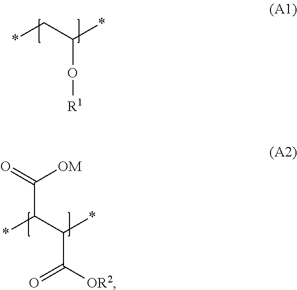

wherein R¹ represents a (C₁ to C₁₈)-alkyl group, R² represents a (C₁ to C₆)-alkyl group, M represents a hydrogen atom or an equivalent of a mono- or polyvalent cation; and 0.1 to 10 wt % of pentasodium triphosphate.

The use of oral and dental hygiene and cleaning agents, including—with respect to the weight thereof—0.1 to 5 wt % of copolymer(s), comprising at least one structural unit of formula (A1) and at least one structural unit of formula (A2)

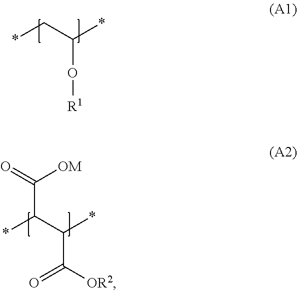

wherein R¹ represents a (C₁ to C₁₈)-alkyl group, R² represents a (C₁ to C₆)-alkyl group, M represents a hydrogen atom or an equivalent of a mono- or polyvalent cation; and 0.1 to 10 wt % of pentasodium triphosphate to reduce the discoloration and/or recoloring of teeth and/or make it easier to remove contaminants from tooth surfaces.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The invention relates to oral and dental hygiene and cleaning agents, including—with respect to the weight thereof—
a) 0.001 to 5 wt % of copolymer(s), comprising at least one structural unit of formula (A1) and at least one structural unit of formula (A2)

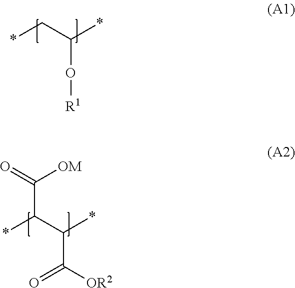

wherein
R¹ represents a (C₁ to C₁₈)-alkyl group,
R² represents a (C₁ to C₆)-alkyl group,
M represents a hydrogen atom or an equivalent of a mono- or polyvalent cation;
b) 0.1 to 10 wt % of pentasodium triphosphate.

The combination of copolymer(s) and pentasodium triphosphate according to the invention promotes the removal of surface stains and prevents the formation of calculus by loosening typical stains from teeth in a more optimized manner. These loosened stains can then be subsequently removed by means of milder cleaning particles.

The combination of copolymer(s) and pentasodium triphosphate according to the invention also leads to reduced mineralization of plaque, which leads to less build-up of calculus.

Oral and dental hygiene agents and oral and dental cleaning agents in the sense of the invention are oral and tooth powders, oral pastes and toothpastes, liquid oral and tooth creams, oral and tooth rinses, and oral and tooth gels. Toothpastes and liquid dental cleaning agents are suitable with preference. For this purpose, the oral and dental hygiene and cleaning agents can exist, for example, in the form of toothpastes, liquid tooth creams, tooth powders, mouthwashes, or chewing mass, e.g., chewing gum. However, they preferably exist as more or less flowable or plastic toothpastes, which are used to clean teeth by using a toothbrush. An additional especially preferred embodiment of the present invention are mouth rinsing solutions and mouthwash, which are used to rinse out the oral cavity.

As a first essential ingredient, the compositions according to the invention contain, with respect to the weight thereof, 0.001 to 5 wt % of copolymer(s), comprising at least one structural unit of formula (A1) and at least one structural unit of formula (A2)

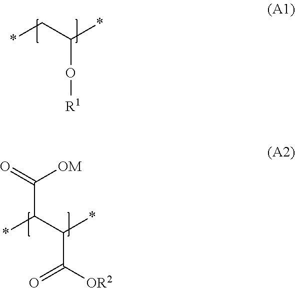

wherein
R¹ represents a (C₁ to C₁₈)-alkyl group,
R² represents a (C₁ to C₆)-alkyl group,
M represents a hydrogen atom or an equivalent of a mono- or polyvalent cation.

According to the formulas above and all formulas below, a chemical bond marked by the symbol * represents a free valence of the corresponding structure fragment.

Examples of (C₁ to C₄)-alkyl groups according to the invention are methyl, ethyl, propyl, isopropyl, butyl, sec-butyl, isobutyl, and tert-butyl.

Examples of (C₈ to C₃₀)-alkyl groups according to the invention are octyl (capryl), decyl (caprinyl), dodecyl (lauryl), tetradecyl (myristyl), hexadecyl (cetyl), octadecyl (stearyl), eicosyl (arachyl), and docosyl (behenyl).

It is preferred according to the invention if the agents according to the invention include as component (b) at least one polymer selected from at least one polymer of the group having polymers of the INCI nomenclature Butyl Ester of PVM/MA Copolymer, Isopropyl Ester of PVM/MA Copolymer, Ethyl Ester of PVM/MA Copolymer.

Corresponding polymers of component a) of the agent according to the invention are, for example, under the trade name Gantrez® ES 425 (copolymer of methyl vinyl ether and the butyl semi-ester of maleic acid; 50 wt % active substance in ethanol; INCI name: Butyl Ester of PVM/MA Copolymer (Ashland)), Gantrez® ES 435 (copolymer of methyl vinyl ether and the butyl semi-ester of maleic acid; 50 wt % active substance in isopropanol; INCI name: Butyl Ester of PVM/MA Copolymer (Ashland)), Gantrez® ES 3351 (copolymer of methyl vinyl ether and the isopropyl semi-ester of maleic acid; 50 wt % active substance in isopropanol; INCI name: Butyl Ester of PVM/MA Copolymer (Ashland)), Gantrez® ES 225 (copolymer of methyl vinyl ether and the ethyl semi-ester of maleic acid; 50 wt % active substance in ethanol; INCI name: Ethyl Ester of PVM/MA Copolymer (Ashland)).

Exceedingly preferred polymers a) have at least one structural unit of formula (A1) and at least one structural unit of formula (A2) in which R¹═R²=M=−H, i.e., copolymers of methyl vinyl ether and maleic acid can be used with particular preference according to the invention. Such copolymers are sold, for example, under the trade name Gantrez® S97 BF (copolymer of methyl vinyl ether and maleic acid (Ashland)).

Especially preferred oral and dental hygiene and cleaning agents according to the invention are characterized in that they include—with respect to the weight thereof—0.005 to 4 wt %, preferably 0.01 to 3 wt %, especially preferably 0.025 to 2 wt %, extraordinarily preferably 0.05 to 1.5 wt %, and particularly 0.1 to 1 wt %, of copolymer(s) of maleic acid and methyl vinyl ether.

It has been found that copolymers a) of a certain molar mass range are especially effective in the combination according to the invention. Especially preferred oral and dental hygiene and cleaning agents according to the invention are therefore characterized in that the copolymer(s), comprising at least one structural unit of formula (A1) and at least one structural unit of formula (A2), has/have molar masses of 500,000 to 5,000,000 daltons, preferably 600,000 to 4,000,000 daltons, more preferably 700,000 to 3,000,000, even more preferably 800,000 to 2,000,000 daltons, and particularly 900,000 to 1,500,000 daltons.

Exceedingly preferred, in turn, is the use of copolymers of methyl vinyl methyl ether and maleic acid, and therefore particularly preferred oral and dental hygiene and cleaning agents are characterized in that they include—with respect to the weight thereof—0.005 to 4 wt %, preferably 0.01 to 3 wt %, especially preferably 0.025 to 2 wt %, extraordinarily preferably 0.05 to 1.5 wt %, and particularly 0.1 to 1 wt %, of copolymer(s) of methyl vinyl ether and maleic acid, which has/have molar masses of 500,000 to 5,000,000 daltons, preferably 600,000 to 4,000,000 daltons, more preferably 700,000 to 3,000,000, even more preferably 800,000 to 2,000,000 daltons, and particularly 900,000 to 1,500,000 daltons.

The agents according to the invention include 0.1 to 10 wt % of pentasodium triphosphate as a second essential ingredient. Pentasodium triphosphate (also sodium tripolyphosphate) is a colorless sodium salt of the triphosphate anion, belongs to the phosphates, and has the empirical formula Na₅P₃O₁₀. The name triphosphate is occasionally used instead of pentasodium triphosphate.

Use within narrow amount ranges is especially effective. Here, preferred oral and dental hygiene and cleaning agents according to the invention are characterized in that they include—with respect to the weight thereof—0.25 to 9 wt %, preferably 0.5 to 8 wt %, especially preferably 0.75 to 7 wt %, extraordinarily preferably 1 to 6 wt %, more preferably 2 to 5.5 wt %, and particularly 3 to 5 wt %, of pentasodium triphosphate.

The oral and dental hygiene and cleaning agents according to the invention can include further ingredients. In this connection, the use of humectants, which prevent drying out in the case of toothpastes, is preferred. In the case of liquid tooth creams having flowable rheology, these serve as a matrix and are used in higher amounts. In mouthwashes and mouth rinses, these alcohols serve as consistency regulators and additional sweeteners.

Preferred here are oral and dental hygiene and cleaning agents according to the invention that include—with respect to the weight thereof—10 to 50 wt %, preferably 12.5 to 45 wt %, especially preferably 15 to 40 wt %, more preferably 17.5 to 35 wt %, and particularly 20 to 29 wt %, of polyhydric alcohol(s) from the group of sorbitol and/or glycerol and/or 1,2-propylene glycol or mixtures thereof.

For certain areas of application, it can be advantageous to use only one of the three ingredients mentioned above. In most cases, sorbitol is preferred. However, in other fields of application, mixtures of two of the three substances or of all three substances can be preferred. A mixture of glycerol, sorbitol, and 1,2-propylene glycol in a weight ratio of 1:(0.5-1):(0.1-0.5) has proven to be especially advantageous here.

In addition to sorbitol and/or glycerol and/or 1,2-propylene glycol, polyhydric alcohols having at least 2 OH groups, preferably mannitol, xylitol, polyethylene glycol, polypropylene glycol, and mixtures thereof are suitable as further polyhydric alcohols. Among these compounds, those with 2 to 12 OH groups and particularly those with 2, 3, 4, 5, 6, or 10 OH groups are preferred.

Examples of polyhydroxy compounds having 2 OH groups are glycol (CH₂(OH)CH₂OH) and other 1,2-diols such as H—(CH₂)ₙ—CH(OH)CH₂OH with n=2, 3, 4, 5, 6, 7, 8, 9, 10, 11, 12, 13, 14, 15, 16, 17, 18, 19, 20. 1,3-diols such as H—(CH₂)ₙ—CH(OH) CH₂CH₂OH with n=1, 2, 3, 4, 5, 6, 7, 8, 9, 10, 11, 12, 13, 14, 15, 16, 17, 18, 19, 20 also can be used according to the invention. The (n,n+1)- or (n,n+2)-diols having non-terminal OH groups can also be used. The polyethylene and polypropylene glycols are also important representatives of polyhydroxy compounds having 2 OH groups. For example, xylitol, propylene glycols, and polyethylene glycols, particularly those having average molecular weights of 200-800, can be used as preferred additional polyhydric alcohols.

Especially preferred is the use of sorbitol, and therefore agents that include no other polyhydric alcohols but sorbitol are especially preferred.

The compositions according to the invention can additionally include at least one polishing agent. In principle, all abrasives known for toothpastes, particularly those that do not include calcium ions, are suitable as polishing agents. Polishing agent components that are suitable with preference are therefore silicic acids, aluminum hydroxide, aluminum oxide, sodium aluminum silicates, organic polymers, or mixtures of such abrasives.

However, calcium-containing polishing components such as chalk, calcium pyrophosphate, and dicalcium phosphate dihydrate can be included in amounts of up to 5 wt % with respect to the entire composition.

The total content of polishing agents lies preferably in the range of 5 to 50 wt % of the dental hygiene agent. Agents preferred according to the invention include polishing agent within narrower amount ranges. Here, oral and dental hygiene and cleaning agents preferred according to the invention are characterized in that they include—with respect to the weight thereof—1 to 25 wt %, preferably 2.5 to 20 wt %, more preferably 5 to 18 wt %, and particularly 7.5 to 16 wt %, of polishing agent.

Especially suitable are toothpastes and liquid dental cleaning agents that include silicic acids as polishing agents. Suitable silicic acids are, for example, gel silicic acids, hydrogel silicic acids, and precipitated silicic acids. Gel silicic acids are produced by reacting sodium silicate solutions with strong, aqueous mineral acids so as to form a hydrosol, aging the hydrosol so as to form a hydrogel, and washing and drying the hydrogel. If the drying is performed under gentle conditions to water contents of 15 to 35 wt %, the hydrogel silicic acids are obtained. By drying to water contents below 15 wt %, an irreversible shrinkage of the previously loose structure of the hydrogel into the tight structure of the xerogel occurs.

A second, especially suitable group of silicic acid polishing agents are the precipitated silicic acids. These are obtained by precipitating silicic acid out of thinned alkali silicate solutions by adding strong acids under conditions in which the aggregation to form the sol and gel cannot occur. Suitable methods for Suitable with preference is a precipitated silicic acid having a BET surface area of 15-110 m²/g, a particle size of 0.5-20 μm, wherein at least 80% wt % of the primary particles should lie under 5 μm, and a viscosity in 30% glycerol-water (1:1) dispersion of 30-60 Pa·s (20° C.) in an amount of 10-20 wt % of the toothpaste. Precipitated silicic acids of this type that are suitable with preference additionally have rounded corners and edges and are available under the trade name Sident® 12 DS (DEGUSSA).

Other precipitated silicic acids of this type are Sident® 8 (Evonik) and Sorbosil® AC 39 (PQ Corporation). These silicic acids are characterized by a lesser thickening effect and a somewhat higher average particle size of 8-14 μm together with a specific surface area of 40-75 m²/g (according to BET) and are particularly well suited for liquid tooth creams. They should have a viscosity (25° C., shear rate D=10 s⁻¹) of 10-100 Pas.

In contrast, toothpastes that have a viscosity significantly greater than 100 Pas (25° C., D=10 s⁻¹) require a sufficiently high proportion of silicic acids having a particle size of less than 5 μm, preferably at least 3 wt % of a silicic acid having a particle size of 1-3 μm. Therefore, in addition to the mentioned precipitated silicic acids, finer-particle thickening silicic acids having a BET surface area of 150-250 m²/g, for example the commercial product Sident® 22S, are also added to such toothpastes.

Aluminum oxide in the form of weakly calcined alumina having a content of—and—aluminum oxide in an amount of approx. 1-5 wt %, for example, can also be included as a further polishing agent component. Such a suitable aluminum oxide is available under the trade name “Poliertonerde P10 feinst” (Giulini Chemie).

Furthermore, all abrasives known for toothpastes, such as sodium aluminum silicates such as zeolite A, organic polymers such as polymethacrylate, or mixtures of these and of the aforementioned abrasives, are suitable as polishing agents.

In summary, oral and dental hygiene and cleaning agents according to the invention that additionally include abrasive substances, preferably silicic acids, aluminum hydroxide, aluminum oxide, calcium pyrophosphate, chalk, dicalcium phosphate dihydrate (CaHPO₄.2H₂O), sodium aluminum silicates, particularly zeolite A, organic polymers, particularly polymethacrylates, or mixtures of these abrastives, preferably in amounts of 1 to 30 wt %, preferably 2.5 to 25 wt %, and particularly 5 to 22 wt %, in each case with respect to the entire agent, are preferred.

Oral and dental hygiene and cleaning agents that are especially preferred according to the invention include exclusively polishing agents from the group of the silicic acids, aluminum hydroxide, aluminum oxide, dicalcium phosphate dihydrate (CaHPO₄.2H₂O), or mixtures of these abrasives. These polishing agents have proven to be especially efficient in solving the problem according to the invention.

Exceedingly preferably, the compositions according to the invention contain, with respect to the weight thereof, 1 to 30 wt % of precipitated silicic acid(s) having a specific surface area according to ISO 5794-1, Annex D, of ≦55 m²/g. The precipitated silicic acids, which have corresponding specific surface areas, are preferably used within narrower amount ranges, and precipitated silicic acids that have even lower specific surface areas according to ISO 5794-1, Annex D, are particularly preferably used. Preferred oral and dental hygiene and cleaning agents according to the invention include 2.5 to 19.5 wt %, preferably 5 to 19 wt %, especially preferably 7.5 to 18.5 wt %, more preferably 8.0 to 18 wt %, and particularly 10.0 to 17.5 wt %, of precipitated silicic acid(s) having a specific surface area according to ISO 5794-1, Annex D, of ≦60 m²/g, preferably of ≦52.5 m²/g, more preferably of ≦49 m²/g, and particularly of ≦47 m²/g.

The oral and dental hygiene and cleaning agents according to the invention can include at least one anionic surfactant as a further essential ingredient.

Suitable anionic surfactants are, for example, linear sodium alkyl sulfates having 12-18 C atoms in the alkyl group. These substances additionally have an enzyme-inhibiting effect on the bacterial metabolism of the plaque. Additional suitable surfactants are alkali salts, preferably sodium salts of alkyl polyglycol ether sulfate having 12-16 C atoms in the linear alkyl group and 2-6 glycol ether groups in the molecule, of linear alkane (C₁₂-C₁₈) sulfonate, of sulfosuccinic acid monoalkyl (C₁₂-C₁₈) esters, of sulfated fatty acid monoglycerides, of sulfated fatty acid alkanolamides, of sulfoacetic acid alkyl (C₁₂-C₁₆) esters, of acyl sarcosines, of acyl taurides, and of acyl isothionates each having 8-18 C atoms in the acyl group.

Further typical examples of anionic surfactants are soaps, alkylbenzene sulfonates, olefin sulfonates, alkyl ether sulfonates, glycerol ether sulfonates, α-methyl ester sulfonates, sulfo fatty acids, alkyl sulfates, fatty alcohol ether sulfates, glycerol ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acyl amino acids, such as acyl lactylates, acyl tartrates, acyl glutamates, and acyl aspartates, alkyl oligoglucoside sulfates, protein fatty acid condensates (particularly plant products based on wheat), and alkyl (ether) phosphates. Provided that the anionic surfactants include polyglycol ether chains, these can have a conventional homolog distribution, but preferably have a restricted homolog distribution.

Oral and dental hygiene and cleaning agents preferred according to the invention are characterized in that they include 0.25 to 4 wt %, preferably 0.5 to 3.5 wt %, more preferably 0.75 to 3 wt %, even more preferably 1 to 2.5 wt %, and particularly 1.25 to 2.2 wt %, of anionic surfactant(s).

The oral and dental hygiene and cleaning agents according to the invention can include at least one amphoteric surfactant as a further ingredient.

Amphoteric surfactants are divided into ampholytic surfactants and zwitterionic surfactants. The term “ampholytic surfactants” is understood to mean surface-active compounds that have both acidic and basic hydrophilic groups and thus behave acidically or basically depending on the condition.

Suitable ampholytic surfactants contain, for example, at least one free amino group (basic hydrophilic group) and at least one —COOH or —SO₃H group (acidic hydrophilic group) in addition to a C₈-C₂₄ alkyl or acyl group and are capable of forming inner salts. Examples of suitable ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids each having approximately 8 to 24 C atoms in the alkyl group. Especially preferred ampholytic surfactants are N-cocoalkylaminopropionate, cocoacylaminoethylaminopropionate, and C₁₂-C₁₈ acyl sarcosine.

Especially preferred oral and dental hygiene and cleaning agents according to the invention are characterized in that they include amphoteric surfactant(s) from the groups of the
N-alkylglycines,
N-alkylpropionic acids,
N-aklylaminobutyric acids,
N-alkyliminodipropionic acids,
N-hydroxyethyl-N-alkylamidopropylglycines,
N-alkyltaurines,
N-alkylsarcosines,
2-alkylaminopropionic acids each having approximately 8 to 24 C atoms in the alkyl group,
alkylaminoacetic acids each having approximately 8 to 24 C atoms in the alkyl group,
N-cocoalkylaminopropionate,
cocoacylaminoethylaminopropionate,
C₁₂-C₁₈ acyl sarcosine,
N-alkyl-N,N-dimethylammoniumglycinate, such as cocoalkyl dimethylammonium glycinate,
N-acylaminopropyl-N,N-dimethylammoniumglycinates, such as cocoacyl aminopropyl dimethylammonium glycinate,
2-alkyl-3-carboxymethyl-3-hydroxyethylimidazolines each having 8 to 18 C atoms in the alkyl or acyl group,
cocoacyl aminoethyl hydroxyethyl carboxymethyl glycinate,
the compounds known under the INCI name Cocamidopropyl Betaine,
the compounds known under the INCI name Disodium Cocoamphodiacetate.

Oral and dental hygiene and cleaning agents that are also preferred contain, as amphoteric surfactants, betaines of formula (Bet-I)

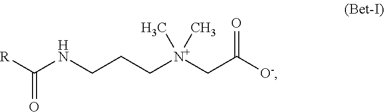

in which R represents a straight-chain or branched, saturated or mono- or polyunsaturated alkyl or alkenyl residue having 8 to 24 carbon atoms.

These surfactants are referred to as amidopropyl betaines in accordance with the INCI nomenclature, wherein the representatives derived from coconut fatty acids are preferred and are referred to as cocamidopropyl betaines. According to the invention, surfactants of formula (Bet-I) that are a mixture of the following representatives are especially preferably used:

H₃C—(CH₂)₇—C(O)—NH—(CH₂)₃N⁺(CH₃)₂CH₂COO⁻

H₃C—(CH₂)₉—C(O)—-NH—(CH₂)₃N⁺(CH₃)₂CH₂COO⁻

H₃C—(CH₂)₁₁—C(O)—NH—(CH₂)₃N⁺(CH₃)₂CH₂COO⁻

H₃C—(CH₂)₁₃—C(O)—NH—(CH₂)₃N⁺(CH₃)₂CH₂COO⁻

H₃C—(CH₂)₁₅—C(O)—NH—(CH₂)₃N⁺(CH₃)₂CH₂COO⁻

H₃C—(CH₂)₇—CH═CH—(CH₂)₇—C(O)—NH—(CH₂)₃N⁺(CH₃)₂CH₂COO⁻

In an additional especially preferred embodiment, an oral and dental hygiene and cleaning agent according to the invention is therefore characterized in that it includes, with respect to the total weight thereof, 0.05 to 5.0 wt %, preferably 0.1 to 3.5 wt %, especially preferably 0.2 to 2.5 wt %, and particularly 0.5 to 2.0 wt %, of amphoteric surfactant(s), preferably cocamidopropyl betaine.

Oral and dental hygiene and cleaning agents, particularly toothpastes, can additionally include further substances that are effective against plaque and/or calculus. Antimicrobial substances and preservatives (plaque) or chelating agents (calculus), for example, are suitable for this purpose.

Oral and dental hygiene and cleaning agents preferred according to the invention are characterized in that they additionally include antiplaque active substances, preferably alkyl pyridinium salts, particularly cetylpyridinium chloride, p-hydroxybenzoic acid methyl, ethyl, or propyl ester, sodium sorbate, sodium benzoate, bromochlorophene, triclosan, hexetidines, phenyl salicylic acid esters, biguanides, such as chlorhexidine, thymol, preferably in amounts of 0.05 to 5 wt %, preferably 0.10 to 2.5 wt %, and particularly 0.30 to 1.5 wt %, in each case with respect to the entire agent.

Substances effective against calculus can be, for example, chelating agents such as ethylenediamine tetraacetic acid and sodium salts thereof, and pyrophosphate salts such as the water-soluble di- or tetra-alkali metal pyrophosphate salts, e.g., Na₄P₂O₇, K₄P₂O₇, Na₂K₂P₂O₇, Na₂H₂P₂O₇, and K₂H₂P₂O₇.

The use of phosphonates is also possible and preferred, wherein compounds of formula (PP-I) have especially proven themselves

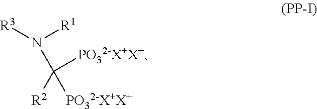

wherein R¹ represents hydrogen or a linear or branched alkyl group having 1 to 6 carbon atoms, R² and R³ represent, independently of each other, a linear or branched alkyl group having 1 to 6 carbon atoms or R² and R³ form a cyclic group having 4 to 8 carbon atoms and optionally having a heteroatom, selected from the group of N, O, and S, and X⁺ represents, independently in each case, H⁺, Li⁺, Na⁺, K⁺, NH₄⁺, ½Mg²⁺, ½Ca²⁺, ½Mn²⁻, ½Zn²⁺, ⅓Al³⁺, ¼Zr⁴⁺.

The compound(s) of formula (PP-I), together with the combination according to the invention, achieve further improved removal of surface stains and prevent the formation of calculus. Especially effective compounds of formula (PP-I) are those in which R¹ represents hydrogen. Preferred compounds of formula (PP-I) are provided in the table below:

| | R¹ | R² | R³ |
|---|---|---|---|
| 1 | —H | —CH₃ | —CH₃ |
| 2 | —H | —CH₃ | —CH₂CH₃ |
| 3 | —H | —CH₃ | —CH₂CH₂CH₃ |
| 4 | —H | —CH₃ | —CH(CH₃)₂ |
| 5 | —H | —CH₃ | —CH₂CH₂CH₂CH₃ |
| 6 | —H | —CH₃ | —CH₂CH(CH₃)₂ |
| 7 | —H | —CH₃ | —CH(CH₃)CH₂CH₃ |
| 8 | —H | —CH₃ | —C(CH₃)₃ |
| 9 | —H | —CH₃ | —(CH₂)₄CH₃ |
| 10 | —H | —CH₃ | —(CH₂)₅CH₃ |
| 11 | —H | —CH₂CH₃ | —CH₃ |
| 12 | —H | —CH₂CH₃ | —CH₂CH₃ |
| 13 | —H | —CH₂CH₃ | —CH₂CH₂CH₃ |
| 14 | —H | —CH₂CH₃ | —CH(CH₃)₂ |
| 15 | —H | —CH₂CH₃ | —CH₂CH₂CH₂CH₃ |
| 16 | —H | —CH₂CH₃ | —CH₂CH(CH₃)₂ |
| 17 | —H | —CH₂CH₃ | —CH(CH₃)CH₂CH₃ |
| 18 | —H | —CH₂CH₃ | —C(CH₃)₃ |
| 19 | —H | —CH₂CH₃ | —(CH₂)₄CH₃ |
| 20 | —H | —CH₂CH₃ | —(CH₂)₅CH₃ |
| 21 | —H | —CH₂CH₂CH₃ | —CH₃ |
| 22 | —H | —CH₂CH₂CH₃ | —CH₂CH₃ |
| 23 | —H | —CH₂CH₂CH₃ | —CH₂CH₂CH₃ |
| 24 | —H | —CH₂CH₂CH₃ | —CH(CH₃)₂ |
| 25 | —H | —CH₂CH₂CH₃ | —CH₂CH₂CH₂CH₃ |
| 26 | —H | —CH₂CH₂CH₃ | —CH₂CH(CH₃)₂ |
| 27 | —H | —CH₂CH₂CH₃ | —CH(CH₃)CH₂CH₃ |
| 28 | —H | —CH₂CH₂CH₃ | —C(CH₃)₃ |
| 29 | —H | —CH₂CH₂CH₃ | —(CH₂)₄CH₃ |
| 30 | —H | —CH₂CH₂CH₃ | —(CH₂)₅CH₃ |
| 31 | —H | —CH(CH₃)₂ | —CH₃ |
| 32 | —H | —CH(CH₃)₂ | —CH₂CH₃ |
| 33 | —H | —CH(CH₃)₂ | —CH₂CH₂CH₃ |
| 34 | —H | —CH(CH₃)₂ | —CH(CH₃)₂ |
| 35 | —H | —CH(CH₃)₂ | —CH₂CH₂CH₂CH₃ |
| 36 | —H | —CH(CH₃)₂ | —CH₂CH(CH₃)₂ |
| 37 | —H | —CH(CH₃)₂ | —CH(CH₃)CH₂CH₃ |
| 38 | —H | —CH(CH₃)₂ | —C(CH₃)₃ |
| 39 | —H | —CH(CH₃)₂ | —(CH₂)₄CH₃ |
| 40 | —H | —CH(CH₃)₂ | —(CH₂)₅CH₃ |
| 41 | —H | —CH₂CH₂CH₂CH₃ | —CH₃ |
| 42 | —H | —CH₂CH₂CH₂CH₃ | —CH₂CH₃ |
| 43 | —H | —CH₂CH₂CH₂CH₃ | —CH₂CH₂CH₃ |
| 44 | —H | —CH₂CH₂CH₂CH₃ | —CH(CH₃)₂ |
| 45 | —H | —CH₂CH₂CH₂CH₃ | —CH₂CH₂CH₂CH₃ |
| 46 | —H | —CH₂CH₂CH₂CH₃ | —CH₂CH(CH₃)₂ |
| 47 | —H | —CH₂CH₂CH₂CH₃ | —CH(CH₃)CH₂CH₃ |
| 48 | —H | —CH₂CH₂CH₂CH₃ | —C(CH₃)₃ |
| 49 | —H | —CH₂CH₂CH₂CH₃ | —(CH₂)₄CH₃ |
| 50 | —H | —CH₂CH₂CH₂CH₃ | —(CH₂)₅CH₃ |
| 51 | —H | —CH₂CH(CH₃)₂ | —CH₃ |
| 52 | —H | —CH₂CH(CH₃)₂ | —CH₂CH₃ |
| 53 | —H | —CH₂CH(CH₃)₂ | —CH₂CH₂CH₃ |
| 54 | —H | —CH₂CH(CH₃)₂ | —CH(CH₃)₂ |
| 55 | —H | —CH₂CH(CH₃)₂ | —CH₂CH₂CH₂CH₃ |
| 56 | —H | —CH₂CH(CH₃)₂ | —CH₂CH(CH₃)₂ |
| 57 | —H | —CH₂CH(CH₃)₂ | —CH(CH₃)CH₂CH₃ |
| 58 | —H | —CH₂CH(CH₃)₂ | —C(CH₃)₃ |
| 59 | —H | —CH₂CH(CH₃)₂ | —(CH₂)₄CH₃ |
| 60 | —H | —CH₂CH(CH₃)₂ | —(CH₂)₅CH₃ |
| 61 | —H | CH(CH₃)CH₂CH₃ | —CH₃ |
| 62 | —H | CH(CH₃)CH₂CH₃ | —CH₂CH₃ |
| 63 | —H | CH(CH₃)CH₂CH₃ | —CH₂CH₂CH₃ |
| 64 | —H | CH(CH₃)CH₂CH₃ | —CH(CH₃)₂ |
| 65 | —H | CH(CH₃)CH₂CH₃ | —CH₂CH₂CH₂CH₃ |
| 66 | —H | CH(CH₃)CH₂CH₃ | —CH₂CH(CH₃)₂ |
| 67 | —H | CH(CH₃)CH₂CH₃ | —CH(CH₃)CH₂CH₃ |
| 68 | —H | CH(CH₃)CH₂CH₃ | —C(CH₃)₃ |
| 69 | —H | CH(CH₃)CH₂CH₃ | —(CH₂)₄CH₃ |
| 70 | —H | CH(CH₃)CH₂CH₃ | —(CH₂)₅CH₃ |
| 71 | —H | —C(CH₃)₃ | —CH₃ |
| 72 | —H | —C(CH₃)₃ | —CH₂CH₃ |
| 73 | —H | —C(CH₃)₃ | —CH₂CH₂CH₃ |
| 74 | —H | —C(CH₃)₃ | —CH(CH₃)₂ |
| 75 | —H | —C(CH₃)₃ | —CH₂CH₂CH₂CH₃ |
| 76 | —H | —C(CH₃)₃ | —CH₂CH(CH₃)₂ |
| 77 | —H | —C(CH₃)₃ | —CH(CH₃)CH₂CH₃ |
| 78 | —H | —C(CH₃)₃ | —C(CH₃)₃ |
| 79 | —H | —C(CH₃)₃ | —(CH₂)₄CH₃ |
| 80 | —H | —C(CH₃)₃ | —(CH₂)₅CH₃ |
| 81 | —H | —(CH₂)₄CH₃ | —CH₃ |
| 82 | —H | —(CH₂)₄CH₃ | —CH₂CH₃ |
| 83 | —H | —(CH₂)₄CH₃ | —CH₂CH₂CH₃ |
| 84 | —H | —(CH₂)₄CH₃ | —CH(CH₃)₂ |
| 85 | —H | —(CH₂)₄CH₃ | —CH₂CH₂CH₂CH₃ |
| 86 | —H | —(CH₂)₄CH₃ | —CH₂CH(CH₃)₂ |
| 87 | —H | —(CH₂)₄CH₃ | —CH(CH₃)CH₂CH₃ |
| 88 | —H | —(CH₂)₄CH₃ | —C(CH₃)₃ |
| 89 | —H | —(CH₂)₄CH₃ | —(CH₂)₄CH₃ |
| 90 | —H | —(CH₂)₄CH₃ | —(CH₂)₅CH₃ |
| 91 | —H | —(CH₂)₅CH₃ | —CH₃ |
| 92 | —H | —(CH₂)₅CH₃ | —CH₂CH₃ |
| 93 | —H | —(CH₂)₅CH₃ | —CH₂CH₂CH₃ |
| 94 | —H | —(CH₂)₅CH₃ | —CH(CH₃)₂ |
| 95 | —H | —(CH₂)₅CH₃ | —CH₂CH₂CH₂CH₃ |
| 96 | —H | —(CH₂)₅CH₃ | —CH₂CH(CH₃)₂ |
| 97 | —H | —(CH₂)₅CH₃ | —CH(CH₃)CH₂CH₃ |
| 98 | —H | —(CH₂)₅CH₃ | —C(CH₃)₃ |
| 99 | —H | —(CH₂)₅CH₃ | —(CH₂)₄CH₃ |
| 100 | —H | —(CH₂)₅CH₃ | —(CH₂)₅CH₃ |

For the compounds of formula (PP-I) in general and for the aforementioned 100 compounds specifically, it is preferred if the 4 cations are defined as follows: 2Na⁺2H⁺.

Extremely preferred compounds of formula (PP-I) are characterized in that the residues R² and R³ form a cyclic group having 4 to 8 carbon atoms and optionally having a heteroatom, selected from the group of N, O, and S.

Exceedingly preferred representatives of this embodiment are stated in the following table:

| | | |
|---|---|---|
| R¹ | R² | R³ |

| | | |
|---|---|---|
| 1 | —H | —(CH₂)₄— |
| 2 | —H | —(CH₂)₅— |
| 3 | —H | —(CH₂)₆— |
| 4 | —H | —(CH₂)₇— |
| 5 | —H | —(CH₂)₈— |
| 6 | —H | —NH—(CH₂)₄— |
| 7 | —H | —(CH₂)—NH—(CH₂)₃— |
| 8 | —H | —(CH₂)₂—NH—(CH₂)₂— |
| 9 | —H | —(CH₂)₃—NH—(CH₂)— |
| 10 | —H | —NH—(CH₂)₅— |
| 11 | —H | —(CH₂)—NH—(CH₂)₄— |
| 12 | —H | —(CH₂)₂—NH—(CH₂)₃— |
| 13 | —H | —(CH₂)₃—NH—(CH₂)₂— |
| 14 | —H | —(CH₂)₄—NH—(CH₂)— |
| 15 | —H | —O—(CH₂)₄— |
| 16 | —H | —(CH₂)—O—(CH₂)₃— |
| 17 | —H | —(CH₂)₂—O—(CH₂)₂— |
| 18 | —H | —(CH₂)₃—O—(CH₂)— |
| 19 | —H | —O—(CH₂)₅— |
| 20 | —H | —(CH₂)—O—(CH₂)₄— |
| 21 | —H | —(CH₂)₂—O—(CH₂)₃— |
| 22 | —H | —(CH₂)₃—O—(CH₂)₂— |
| 23 | —H | —(CH₂)₄—O—(CH₂)— |
| 24 | —H | —S—(CH₂)₄— |
| 25 | —H | —(CH₂)—S—(CH₂)₃— |
| 26 | —H | —(CH₂)₂—S—(CH₂)₂— |
| 27 | —H | —(CH₂)₃—S—(CH₂)— |
| 28 | —H | —S—(CH₂)₅— |
| 29 | —H | —(CH₂)—S—(CH₂)₄— |
| 30 | —H | —(CH₂)₂—S—(CH₂)₃— |
| 31 | —H | —(CH₂)₃—S—(CH₂)₂— |
| 32 | —H | —(CH₂)₄—S—(CH₂)— |

Likewise for the aforementioned 32 compounds, it is preferred if the 4 cations are defined as follows: 2Na⁺2H⁺.

A compound of formula (PP-I) to be used exceedingly preferably is azacycloheptane diphosphonic acid. Extremely preferred oral and dental hygiene and cleaning agents according to the invention are therefore characterized in that they include—with respect to the weight thereof—0.025 to 4.5 wt %, preferably 0.05 to 4 wt %, especially preferably 0.1 to 3 wt %, extraordinarily preferably 0.15 to 2 wt %, more preferably 0.25 to 1.5 wt %, and particularly 0.5 to 1 wt %, of compound(s) of formula (PP-Ia)

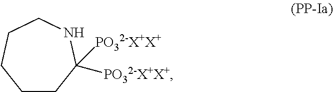

wherein X⁺ represents, independently in each case, H⁺, Li⁺, Na⁺, K⁺, NH₄⁺, ½Mg²⁺, ½Ca²⁺, ½Mn²⁺, ½Zn²⁺, ⅓Al³⁺, ¼Zr⁴⁺.

Here also, it is preferred if the 4 cations are defined as follows: 2Na⁺2H⁺, and therefore extremely preferred oral and dental hygiene and cleaning agents according to the invention are characterized in that they include—with respect to the weight thereof—0.025 to 4.5 wt %, preferably 0.05 to 4 wt %, especially preferably 0.1 to 3 wt %, extraordinarily preferably 0.15 to 2 wt %, more preferably 0.25 to 1.5 wt %, and particularly 0.5 to 1 wt %, of disodium azacycloheptane diphosphonate.

The dental hygiene agents according to the invention can be improved in the organoleptic properties thereof by, for example, adding flavoring oils and sweeteners. All natural and synthetic flavorings common for oral and dental hygiene agents can be used as flavoring oils. Natural flavorings can be included both in the form of the natural essential oils isolated from drugs and in the form of the individual components isolated therefrom. Suitable flavorings as are, for example, peppermint oil, spearmint oil, eucalyptus oil, aniseed oil, fennel oil, caraway oil, menthyl acetate, cinnamaldehyde, anethole, vanillin, thymol, and mixtures of these components.

Suitable sweeteners are, for example, saccharin sodium, sodium cyclamate, sucrose, lactose, Meltose, fructose, maltose, and dextrose.

It has been found that the action of the combination according to the invention can be expanded to include a further effect against halitosis and in particular against gingivitis and periodontitis if zinc salts are used.

Agents preferred according to the invention therefore include at least one zinc salt, preferably in amounts of 0.001 to 5 wt %, with respect to the entire agent, wherein especially preferred oral and dental hygiene and cleaning agents are characterized in that they include 0.001 to 5 wt %, preferably 0.02 to 2.5 wt %, especially preferably 0.03 to 2.0 wt %, more preferably 0.04 to 1.5 wt %, even more preferably 0.05 to 1.0 wt %, even more preferably 0.06 to 0.5 wt %, and particularly 0.07 to 0.25 wt %, of at least one zinc salt.

Here—as always if no other specifications are made in the present document—the amount specifications relate to the entire agent, i.e., the finished oral and dental hygiene and cleaning agent.

According to the invention, both inorganic and organic salts of zinc can be used. In addition to the insoluble inorganic zinc salts, i.e., salts that have a solubility below 100 mg/L (20° C.), preferably below 10 mg/L (20° C.), particularly no solubility (20° C.) (for example, zinc oxide), the soluble inorganic zinc salts, i.e., salts that have a solubility above 100 mg/L, preferably above 500 mg/L, especially preferably above 1 g/L, and particularly above 5 g/L, are a preferred constituent of agents according to the invention in the context of the present application. The preferred soluble inorganic salts include zinc bromide, zinc chloride, zinc iodide, zinc nitrate, and zinc sulfate.

Agents especially preferred according to the invention include at least one zinc salt from the table below:

| Zinc salt | Solubility | |
|---|---|---|
| Zinc acetate dihydrate | 430 g/l | (20° C.) |
| Zinc acetylacetonate | 4 g/l | (20° C.) |
| Zinc bromide | 820 g/l | (25° C.) |
| Zinc chloride | 4320 g/l | (25° C.) |
| Zinc gluconate | 100 g/l | (20° C.) |
| Zinc hydroxycarbonate | Nearly insoluble | (20° C.) |
| Zinc iodide | 4500 g/l | (20° C.) |
| Zinc nitrate hexahydrate | 1843 g/l | (20° C.) |
| Zinc nitrate tetrahydrate | Slightly soluble | (20° C.) |
| Zinc oxide | Insoluble | |
| Zinc stearate | 0.9 mg/l | (20° C.) |
| Zinc sulfate heptahydrate | 960 g/l | (20° C.) |
| Zinc sulfate monohydrate | ~350 g/l | (20° C.) |

The spectrum of the zinc salts of organic acids, preferably of organic carboxylic acids, preferred according to the invention ranges from salts that are insoluble in water, i.e., that have a solubility below 100 mg/L, preferably below 10 mg/L, particularly no solubility, to salts that have a solubility in water above 100 mg/L, preferably above 500 mg/L, especially preferably above 1 g/L, and particularly above 5 g/L (all solubilities at 20° C. water temperature). The first group of zinc salts includes, for example, zinc citrate, zinc laureate, zinc oleate, zinc oxalate, zinc tartrate, and zinc stearate. The group of the soluble organic zinc salts includes, for example, zinc acetate, zinc acetylacetonate, zinc benzoate, zinc formate, zinc lactate, zinc gluconate, zinc valerate, and the zinc salt of p-toluenesulfonic acid.

It has been found that zinc sulfate is a zinc salt to be used with particular preference. Zinc sulfate forms several hydrates. The heptahydrate is formed in the form of colorless, glass-shiny, columnar, rhomboid crystals from saturated aqueous solution. A conversion to ZnSO₄.6H₂O occurs above 39° C., and only ZnSO₄.H₂O is still present at 70° C.; the last H₂O molecule escapes at 240° C. Interestingly, the zinc sulfates' effect according to the invention increases with the crystal water content thereof, and therefore the heptahydrate is an especially preferred compound.

Oral and dental hygiene and cleaning agents preferred according to the invention therefore include zinc sulfate, preferably zinc sulfate heptahydrate.

Agents according to the invention can be formulated as toothpastes or tooth creams. The present invention also relates to the use of agents according to the invention to clean teeth by means of manually operated or electric toothbrushes. In the case of electric toothbrushes, the agents according to the invention have the further advantage that they are effective even in small amounts and also do not impair the mechanical system of the electric brush head.

The present invention also relates to a method for cleaning teeth, characterized in that an agent according to the invention is applied to the brush head of a manually operated or electric toothbrush and the teeth are brushed by means of the toothbrush.

The present invention also relates to a method for cleaning teeth, characterized by the following steps:
a) providing a toothbrush, the brush head of which can be set into motion;
b) applying 0.5 to 5 g of an agent according to the invention to the brush head;
c) brushing the teeth with the agent according to one of claims 1 to 8 for 30 to 300 seconds by using the brush head set into motion.

The present invention also relates to the use of oral and dental hygiene and cleaning agents, including—with respect to the weight thereof—
a) 0.1 to 5 wt % of copolymer(s), comprising at least one structural unit of formula (A1) and at least one structural unit of formula (A2)

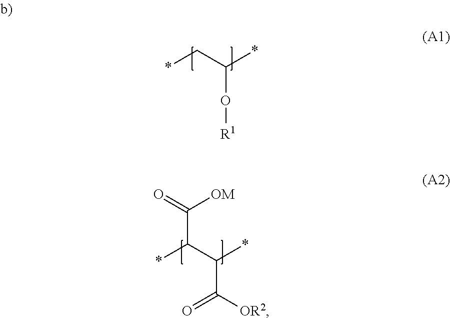

wherein
R¹ represents a (C₁ to C₁₈)-alkyl group,
R² represents a (C₁ to C₆)-alkyl group,
M represents a hydrogen atom or an equivalent of a mono- or polyvalent cation;
c) 0.1 to 10 wt % of pentasodium triphosphate to
   reduce the discoloration and/or recoloring of teeth and/or
   make it easier to remove contaminants from tooth surfaces
   prevent the reforming of calculus in an optimized manner.

That which was said in regard to the agents according to the invention applies, mutatis mutandis, to preferred embodiments of the methods and uses according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An oral and dental hygiene and cleaning agent, comprising—with respect to the weight thereof—
a) 0.001 to 5 wt % of copolymer(s), comprising at least one structural unit of formula (A1) and at least one structural unit of formula (A2) 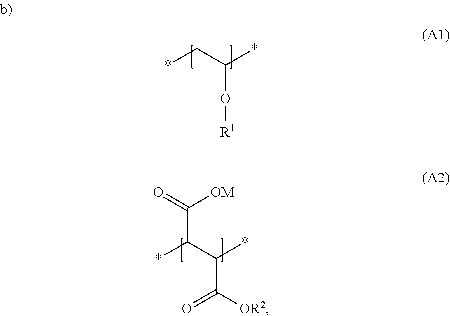
wherein
R¹ represents a (C₁ to C₁₈)-alkyl group,
R² represents a (C₁ to C₆)-alkyl group,
M represents a hydrogen atom or an equivalent of a mono- or polyvalent cation;
c) 0.1 to 10 wt % of pentasodium triphosphate.

2. The oral and dental hygiene and cleaning agent according to claim 1, wherein the copolymer(s) comprises 0.002 to 4 wt % of the total weight of the agent.

3. The oral and dental hygiene and cleaning agent according to claim 1, wherein the copolymer(s) comprises 0.003 to 3 wt % of the total weight of the agent.

4. The oral and dental hygiene and cleaning agent according to claim 1, wherein the copolymer(s) comprises 0.005 to 1 wt % of the total weight of the agent.

5. The oral and dental hygiene and cleaning agent according to claim 1, wherein the copolymer(s) comprises 0.01 to 0.5 wt % of the total weight of the agent.

6. The oral and dental hygiene and cleaning agent according to claim 1, wherein the copolymer(s) is a copolymer of maleic acid and methyl vinyl ether.

7. The oral and dental hygiene and cleaning agent according to claim 1, wherein the copolymer(s) comprising at least one structural unit of formula (A1) and at least one structural unit of formula (A2) has/have molar masses of 500,000 to 5,000,000 daltons.

8. The oral and dental hygiene and cleaning agent according to claim 1, wherein the copolymer(s) comprising at least one structural unit of formula (A1) and at least one structural unit of formula (A2) has/have molar masses of 600,000 to 4,000,000 daltons.

9. The oral and dental hygiene and cleaning agent according to claim 1, wherein the copolymer(s) comprising at least one structural unit of formula (A1) and at least one structural unit of formula (A2) has/have molar masses of 900,000 to 1,500,000 daltons.

10. The oral and dental hygiene and cleaning agent according to claim 1, wherein the pentasodium triphosphate comprises 0.25 to 9 wt % of the total cleaning agent.

11. The oral and dental hygiene and cleaning agent according to claim 1, wherein the pentasodium triphosphate comprises 0.5 to 8 wt % of the total cleaning agent.

12. The oral and dental hygiene and cleaning agent according to claim 1, wherein the pentasodium triphosphate comprises 2 to 5.5 wt % of the total agent.

13. The oral and dental hygiene and cleaning agent according to claim 1, further comprising 0.25 to 4 wt % of at least one anionic surfactant.

14. The oral and dental hygiene and cleaning agent according to claim 1, further comprising 0.75 to 3 wt % of at least one anionic surfactant.

15. The oral and dental hygiene and cleaning agent according to claim 1, further comprising 1.25 to 2.2 wt % of at least one anionic surfactant.

16. The oral and dental hygiene and cleaning agent according to claim 1, wherein the oral and dental hygiene and cleaning agent further comprises 0.25 to 4 wt % of sodium dodecyl sulfate based on the total weight of agent.

17. The oral and dental hygiene and cleaning agent according to claim 1, wherein the oral and dental hygiene and cleaning agent further comprises 2.5 to 19.5 wt % of precipitated silicic acid(s) having a specific surface area according to ISO 5794-1, Annex D, of ≦60 m²/g.

18. The oral and dental hygiene and cleaning agent according to claim 1, wherein the oral and dental hygiene and cleaning agent further comprises 10.0 to 17.5 wt % of precipitated silicic acid(s) having a specific surface area according to ISO 5794-1, Annex D, of ≦47 m²/g.

19. The oral and dental hygiene and cleaning agent according to one of claim 1, wherein the oral and dental hygiene and cleaning agent further comprises 0.001 to 5 wt % of at least one zinc salt.

20. A method for cleaning teeth, characterized by the following steps:
d) providing a toothbrush, the brush head of which can be electrically set into motion;
e) applying 0.5 to 5 g of an agent according to claim 1 to the brush head,
f) brushing the teeth with the agent for 30 to 300 seconds by using the brush head electrically set into motion.
